(19) United States
(12) Patent Application Publication
(10) Pub. No.: US 2021/0183512 A1
(43) Pub. Date: Jun. 17, 2021

(54) SYSTEMS, APPARATUS, AND METHODS TO MONITOR PATIENTS AND VALIDATE MENTAL ILLNESS DIAGNOSES

(71) Applicant: The Nielsen Company (US), LLC, New York, NY (US)

(72) Inventor: Teresa Van Dusen, Richardson, TX (US)

(21) Appl. No.: 16/713,754

(22) Filed: Dec. 13, 2019

(51) Int. Cl. G16H 50/20; G16H 50/70; G16H 50/30; G16H 40/67; G16H 10/60; G06Q 50/26; G06F 16/28; H04B 1/3827; G08B 21/04; A61B 5/00; A61B 5/16

(52) U.S. Cl. CPC G16H 50/20; A61B 5/168; G16H 50/30; G16H 40/67; G16H 10/60; G06Q 50/265; G06F 16/285; H04B 1/385; G08B 21/0453; G08B 21/0423; A61B 5/7282; A61B 5/7275; A61B 5/165; A61B 5/746; G16H 50/70

(57) ABSTRACT

Systems, apparatus, and methods are disclosed to monitor patients and validate mental illness. An example apparatus includes an identifier to identify a population behavioral baseline based on a patient-specific demographic data, the patient-specific demographic data retrieved from at least one of a third-party subscriber data or an audience measurement entity data, an evaluator to compare a patient behavioral baseline to the population behavioral baseline to determine a correlation between the patient behavioral baseline and the population behavioral baseline, and a validator to identify the diagnosis as valid when the correlation is low.

### FIELD OF THE DISCLOSURE

This disclosure relates generally to medical diagnosis, and, more particularly, to systems, apparatus, and methods to monitor patients and validate mental illness diagnoses.

### BACKGROUND

A mental illness diagnosis can be made based on a mental health professional's (e.g., therapist, psychiatrist, psychologist, etc.) observation of an individual in order to identify existing symptoms, in combination with a health professional's knowledge of the individual's medical history and life events. An important aspect of the diagnosis relates to changes in the individual's behavior, as well as determinations as to whether any symptoms related to a mental illness are not a result of a physical condition. An accurate mental illness diagnosis relies on careful assessment and identification of a cause of behavioral changes identified over a period of time during which the individual can be observed.

### BRIEF DESCRIPTION OF THE DRAWINGS

FIG. 1 is a block diagram illustrating an example operating environment, constructed in accordance with teachings of this disclosure, in which a patient monitoring system is implemented for patient monitoring and validation of a mental illness diagnosis.

FIG. 2 is a block diagram of an example implementation of the patient monitoring system of FIG. 1.

FIG. 3 is a flowchart representative of computer readable instructions that may be executed to implement elements of the example patient monitoring system of FIGS. 1-2.

FIG. 4 is a flowchart representative of computer readable instructions that may be executed to implement elements of the example patient monitoring system of FIGS. 1-2, the flowchart representative of instructions used to collect patient behavioral data.

FIG. 5 is a flowchart representative of computer readable instructions that may be executed to implement elements of the example patient monitoring system of FIGS. 1-2, the flowchart representative of instructions used to identify population behavioral data.

FIG. 6 is a flowchart representative of computer readable instructions that may be executed to implement elements of the example patient monitoring system of FIGS. 1-2, the flowchart representative of instructions used to validate a medical diagnosis.

FIG. 7 is a flowchart representative of computer readable instructions that may be executed to implement elements of the example patient monitoring system of FIGS. 1-2, the flowchart representative of instructions used to activate a support system.

FIG. 8 is a block diagram of an example processing platform structured to execute the instructions of FIGS. 3-7 to implement the example patient monitoring system of FIGS. 1-2.

The figures are not to scale. In general, the same reference numbers will be used throughout the drawing(s) and accompanying written description to refer to the same or like parts. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### DETAILED DESCRIPTION

Mental illness can be diagnosed using several methods, including, for example, a physical and laboratory-based examination to rule out physical problems causing metal illness-related symptoms, and/or a psychological evaluation to determine behavioral patterns and an overall emotional state of the individual. An accurate diagnosis is the foundation for receiving appropriate and effective treatment. In examples disclosed herein, an understanding of the symptoms is made possible through a diversity of information regarding the patient's medical history and behavioral profile. Defining symptoms of mental illness are detailed in the American Psychiatric Association's Diagnostic and Statistical Manual of Mental Disorders (DSM-5), which is used by mental health professionals to diagnose mental conditions. Mental illnesses are classified into a variety of categories, including, for example, neurodevelopmental disorders, schizophrenia spectrum disorders, bipolar disorders, depressive disorders, anxiety disorders, trauma- and stressor-related disorders, and personality disorders, among others. Treatment can be determined based on the type of mental illness, severity, and treatment approach that is most appropriate for a patient (e.g., based on existing medical conditions, personal preferences, etc.). In some situations, for patients with severe mental illness, the treatment plan is formed and adhered to based on the involvement of a number of healthcare professions, which can include, for example, a primary care doctor, a nurse practitioner, a psychiatrist, a psychotherapist, a pharmacist, and/or a social worker. In some instances, psychiatric medication (e.g., mood-stabilizing medication, antipsychotic medication, antidepressants, etc.) can significantly improve mental illness-related symptoms. However, at times, psychiatric medication does not provide a cure, which may necessitate ongoing patient-oriented treatment. Ongoing patient-oriented treatment seeks to create a long-term reduction in patient symptoms and allow the patient and/or caregiver to better manage any potential triggers, which can, for example, exacerbate the symptoms. In some examples, such an approach can include use of psychotherapy, permitting the patient to talk about their condition and learn techniques to manage their illness. In severe cases, the patient can receive care in a psychiatric hospital, especially when the patient is not able to care for themselves or is in immediate danger of harming themselves or someone else.

Mental illness is a common occurrence given that 1 in 5 adults have a mental illness in any given year, with the effects being either temporary or long lasting. Untreated mental illness can result in severe emotional, behavioral, and/or physical health problems, including the risk of self-harm (e.g., suicide). In some examples, patients can have multiple mental health disorders at the same time, making the treatment and management of their condition more complex. Given the range of potential mental health disorders, an accurate diagnosis can be difficult to establish. This difficulty may result in misdiagnoses. Some frequently misdiagnosed mental health disorders include bipolar disorder, depression, borderline personality disorder, attention deficit hyperactivity disorder (ADHD), post-traumatic stress disorder (PTSD), and anxiety. Treatment including, for example, administration or prescription of pharmaceuticals may cause harm to a misdiagnosed patient. For example, a patient receiving a diagnosis of a major depressive disorder can be prescribed daily doses of a selective serotonin reuptake inhibitor (SSRI)-based antidepressant. If the patient is misdiagnosed, such medication will not be appropriate for the patient's condition and can cause new psychological and physical symptoms (e.g., an unstable mood). A greater level of detail about the patient's condition can assist the mental health professional in making a correct diagnosis.

Also, in some situations, a patient may not themselves recognize some of the symptoms that are related to their condition (e.g., quality of sleep, levels of energy, etc.). For example, a bipolar disorder can go unrecognized, and the patient may instead be diagnosed with a major depressive disorder based on the patient's self-reporting. Statistics indicate that individuals with bipolar disorder, for example, can spend years receiving treatment for other conditions, with many being incorrectly identified as having depression, resulting in prescriptions of antidepressants or sleeping pills. Antidepressants heighten risk of mania, increase frequency of mood switching, and intensify episodes of depression. Given that patients with bipolar disorder have the highest rates of suicide of any psychiatric illness, misdiagnosis is particularly dangerous for the well-being of such patients. Such misdiagnoses can occur when a patient does not report certain behaviors that are symptomatic of mental illness, especially if such information is not explicitly requested. In some examples, patients may not feel comfortable admitting certain symptoms to their health provider due to shame or embarrassment as a result of self-consciousness. Not only does mental health misdiagnosis prevent individual from receiving proper treatment, but mental health misdiagnosis also threatens their emotional and behavioral function due to lack of treatment or treatment with unnecessary pharmacological therapies. In fact, each additional mood episode can instigate neurological changes that further complicate treatment and recovery.

Steps to reduce misdiagnosis include performing a comprehensive psychological assessment and involving observers familiar with the patient behavior (e.g., family members who can participate in the diagnostic process to provide an additional perspective of a patient's behavior pattern). Another aspect of establishing an accurate diagnosis includes creating an environmental in which the patient feels comfortable sharing any experiences, thoughts, and/or emotions that are necessary to understand their state of mind and behavior. Such comprehensive assessments can be performed in high-quality residential treatment environments where ongoing monitoring can take place to observe evolving symptoms and medication responses. However, such environments are not accessible to many patients, with most relying on formal psychological assessments that can be performed in any setting. Additionally, it can be challenging to determine whether patient behavior falls within a behavioral norm for a given demographic, given that societal norms of behavior change over time and based on generational level.

The example systems, apparatus, and methods disclosed herein permit verification of a mental illness diagnosis based on patient behavioral and demographic data. Furthermore, example technical solutions disclosed herein monitor patients at high risk to establish a comprehensive assessment of their risk of self-harm based on behavioral data, clinical data, and environmental data. Examples disclosed herein leverage a various number of techniques obtaining patient-based information to monitor patients and assess the accuracy of a mental illness diagnosis based on comparison to an established population-derived behavioral baseline. Example technical solutions establish behavior baseline of the population according to demographic data based on behavioral data, which can be collected by various third-party service providers (e.g., Facebook, Twitter, other websites, internet service providers, telecommunication companies, etc.), as well as marketing research and/or healthcare research companies.

In examples disclosed herein, audience measurement entity panel meter data (e.g., derived from registered panelists) (e.g., data obtained from an AME such as The Nielsen Company (US), LLC) can be leveraged to identify a population behavioral baseline in order to compare the population behavioral baseline with patient behavioral data as part of validating a mental illness diagnosis. AMEs monitor viewing of media presented by media devices, which can be used to extrapolate information relevant to establishing a population and/or patient-based behavior baseline (e.g., demographic-based media content exposure, media content preferences, and/or selections). For example, AMEs can perform measurements to determine the number of people (e.g., an audience) that engages in viewing television, listening to radio stations, and/or browsing websites via on-device meters (ODMs) used to monitor usage of cellphones, tablets (e.g., iPads™), PDAs, laptop computers, and/or other computing devices of individuals including those who volunteer to be part of a panel (e.g., panelists). Panelists are users who have provided demographic information at the time of registration into a panel, allowing their demographic information to be linked to the media they choose to listen to or view, which may be monitored via one or more meters. As a result, the panelists (e.g., the audience) represent a statistically significant sample of the large population (e.g., the census) of media consumers. In some examples, an AME may extrapolate ratings metrics and/or other audience measurement data for a total media viewing audience from a relatively small sample of panel homes (e.g., homes with individuals (e.g., panelists) who have agreed to be monitored and have provided their demographic information to the AME). In some examples, the media consumption information can be used to determine population-specific behavior within a given demographic categorization. Examples disclosed herein classify a patient according to their demographic data, which may correspond to demographic categorizations of the AME. The patient behavior may be compared with behavior baseline determined by the AME demographic categorization for the same or similar demographic categories. The baseline being updated periodically, aperiodically, during, and/or after data collection by the AME from the population at large and/or specific panelist(s).

Following an initial patient assessment period, the examples disclosed herein determine the level of risk associated with a given patient behavior (e.g., high level of risk would correspond to the potential of the patient to commit suicide). A support system can be activated to monitor the patient (e.g., via wearable devices, smart phone applications, and/or social robot-based interactions) for comprehensive patient assessment (e.g., environmental data, behavioral data, and/or clinical data). In some examples, the existing healthcare support network can configure trigger warnings based on human observations and system-assessed behavioral patterns (e.g., insomnia, specific physiological symptoms, etc.). In some examples, wearable devices can be engaged to collect environmental data on patient location, ambient and/or patient body temperature, environmental sounds and/or patient noises, a web browsing history, an amount of screen time on a computing device, etc. Furthermore, the active support system can be designed to alleviate the burden on the patient and their human support system by reminding the patient of medication time/dosage, recording journaling information, involving patient in exercises, etc. Throughout the patient monitoring period, collected data is compared to patient and/or population behavioral baseline data to identify sudden changes in behavior (e.g., poor sleeping habits) and environment (e.g., dangerous locations). Identification of such triggers permits increased efficiency in response time and earlier identification of signs indicative of a higher probability of patient-initiated acts that can lead to harm including, for example, suicide. The examples disclosed herein are not limited to mental illness diagnosis validation or monitoring of patients specifically at a high risk of patient suicide, but can be applied for purposes of validating other medical diagnosis where a behavioral analysis increases the accuracy of a given diagnosis.

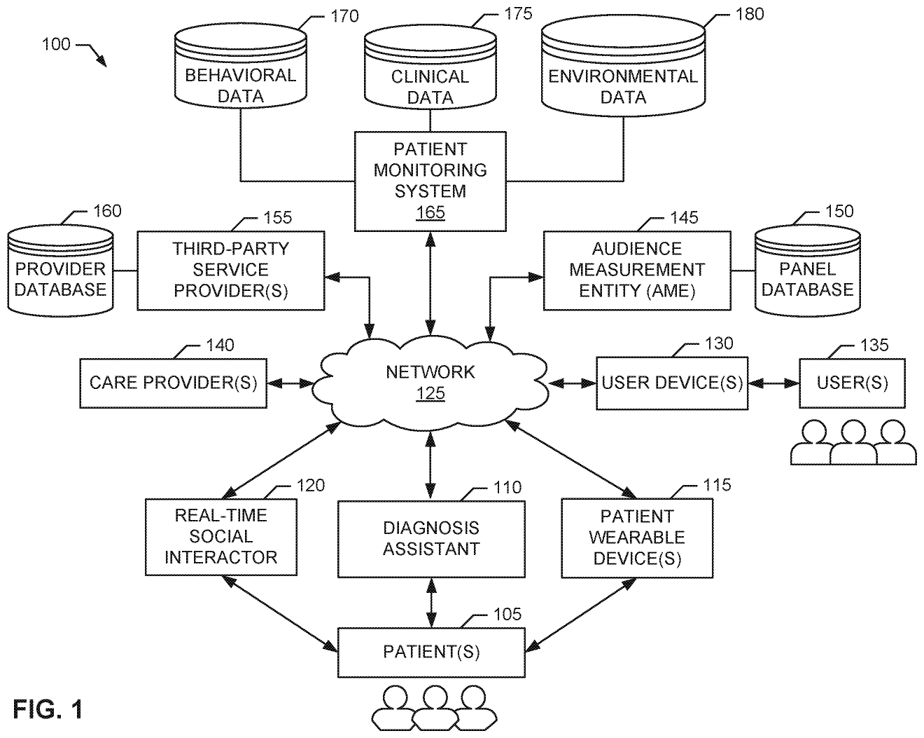

FIG. 1 is a block diagram illustrating an example operating environment 100, constructed in accordance with teachings of this disclosure, in which a patient monitoring system is implemented for patient monitoring and validation of a mental illness diagnosis. The example operating environment 100 of FIG. 1 includes example patient(s) 105, an example diagnosis assistant 110, example patient wearable device(s) 115, an example real-time social interactor 120, an example network 125, example user device(s) 130, example user(s) 135, example care provider(s) 140, an example audience measurement entity (AME) 145, example third-party service provider(s) 155, and example patient monitoring system 165. The AME 145 includes an example panel database 150. The third-party service provider(s) 155 includes an example provider database 160. The patient monitoring system 165 includes an example behavioral database 170, an example clinical database 175, and an example environment database 180.

Patient(s) 105 can include any individuals who are given an initial diagnosis of a mental illness (e.g., a disorder that affects the individual mood, thinking, and/or behavior, including depression, anxiety disorder, schizophrenia, addictive behaviors, etc.). Patient(s) 105 can include patients who are in a residential treatment environment, under home-based care (e.g., not enrolled in a dedicated treatment/monitoring program), and/or are in an initial assessment period to establish a diagnosis. The patient(s) 105 interaction can include interaction with patient wearable device(s) 115 (e.g., that can provide patent-specific behavioral, physiological, and/or environmental data), the diagnosis assistant 110, and/or the real-time social interactor 120.

Diagnosis assistant 110 uses a digital representation of the Diagnostic & Statistical Manual of Mental Disorders (DSM-5), a known diagnostic tool in the mental health professional community, for purposes of referencing established symptoms of mental illnesses. The diagnosis assistant 110 can be used, in some examples, as part of a routine assessment by a mental health professional to assist in establishing an initial mental illness diagnosis. The diagnosis assistant 110 can receive an input from the health care professionals of symptoms and/or behaviors that are used to establish an initial diagnosis. In some examples, the symptoms and behaviors also are used as part of the patient monitoring system 165 to compare against behavioral patterns determined using the monitoring system 165. Also, in some examples, the symptoms and behaviors are compared to the behavior that may be reported by the patient and/or family member to the health care provider during the initial assessment and establishment of the medical diagnosis. The diagnosis assistant 110 can communicate input information to the patient monitoring system 165 via the example network 125.

Patient wearable device(s) 115 include any devices used by the patient to collect behavioral data, clinical data, and/or environmental data. For example, the device(s) 115 can include any wearable devices able to track user activity (e.g., data obtained from use of a computing device, including screen time usage, web browsing history, etc.), location and/or environmental characteristics (e.g., geographic location, location-based sounds, speed, acceleration, smells, temperature, etc.) and/or physiological condition (e.g., body temperature, heart rate, electrocardiographic data, sleep pattern/habits, electroencephalographic data, electromyographic data, electrooculographic data, functional magnetic resonance imaging data, eye gaze direction, pupillary response/eye dilation, galvanic skin response, etc.). For example, when the patient monitoring system 165 collects patient behavioral data as part of a medical diagnosis validation process, the patient monitoring system 165 can store data recorded by the patient wearable device(s) 115 in the behavioral database 170, clinical database 175, and/or environmental database 180. In some examples, the patient wearable device(s) 115 can be used when activating a support system once a high risk to the patient is established (e.g., high risk identification of self-harm and/or indications of potential or actual suicide attempts). In such examples, the patient wearable device(s) 115 can be used to determine presence of parameters that are indicative of high-risk to the patient and/or monitoring of deviations from normal patient behavior (e.g., disrupted sleep, presence of patient in an environment that is dangerous, sudden changes in blood pressure, etc.). In some examples, the wearable device(s) can include a smartphone (e.g., an Apple® iPhone®, a Motorola™ Moto X™, a Nexus 5, an Android™ platform device, etc.), a smartwatch, a headset, a ring, a bracelet, clothing embedded with one or more sensors, and/or any type of wearable activity tracker and/or other type of sensor used to gather data disclosed herein. In some examples, the wearable device(s) are not worn but are disposed in proximity to the patient to gather the data disclosed herein. The patient wearable device(s) 115 can communicate data to the patient monitoring system 165 via the example network 125.

Real-time social interactor 120 can include any device that can interact with the patient(s) 105 to perform patient interaction-based assessments that would otherwise be performed by a health care professional (e.g., reminding patient to take medication, reminding patient of appointments, recoding patient journaling activity). The real-time social interactor 120 can be used in a facility-based setting (e.g., hospital, care center, etc.) or in a home-based setting, for purposes of alleviating the burden on care givers. In some examples, the social interactor 120 can be a dedicated robot that interacts with the patient. In some examples, the social interactor 120 can be implemented via one or more applications accessible to the patient via an electronic device such as a smartphone, a tablet, and/or a laptop. In some examples, the real-time social interactor 120 engages the patient with different activities to promote patient interaction (e.g., games, exercise routines, mediation sessions, etc.). The real-time social interactor 120 can be useful for patients who are in a state of disengagement from normal interactions with other individuals, such that interaction with the real-time social interactor 120 can assist in engaging these patients in activities that a healthcare professional or another caretaker may have difficulty accomplishing. In some examples, the real-time social interactor 120 can also be used to monitor patient response and/or behavior in order to collect additional data on patient behavior. For example, a patient interacting with a real-time social interactor 120 may, in some examples, reveal more information when asked about certain symptoms as compared to when being evaluated by a human health care professional (e.g., withholding certain symptoms and/or information that could be pertinent to the diagnosis). The real-time social interactor 120 can communicate data to the patient monitoring system 165 via the example network 125.

Network 125 may be implemented using any suitable wired and/or wireless network(s) including, for example, one or more data buses, one or more Local Area Networks (LANs), one or more wireless LANs, one or more cellular networks, the Internet, etc. As used herein, the phrase “in communication,” including variances thereof, encompasses direct communication and/or indirect communication through one or more intermediary components and does not require direct physical (e.g., wired) communication and/or constant communication, but rather additionally includes selective communication at periodic or aperiodic intervals, as well as one-time events.

User device(s) 130 can be stationary or portable computers, handheld computing devices, smart phones, Internet appliances, and/or any other type of device that may be connected to the Internet and capable of presenting media. For example, the user device(s) 130 can include a smartphone (e.g., an Apple® iPhone®, a Motorola™ Moto X™, a Nexus 5, an Android™ platform device, etc.) or a laptop computer. However, any other type of device may additionally or alternatively be used such as, for example, a tablet (e.g., an Apple® iPad™, a Motorola™, Xoom™, etc.), a desktop computer, a camera, an Internet compatible television, a smart TV, etc. The user device(s) 130 are used to access (e.g., request, receive, render and/or present) online media provided, for example, by a web server. For example, user(s) 135 can execute a web browser on the user device(s) 130 to request streaming media (e.g., via an HTTP request) from a media hosting server. The web server can be any web browser used to provide media content (e.g., YouTube) that is accessed, through the network 125, by the example user(s) 135 on example user device(s) 130. The user device(s) 130 can include any devices that are used by the population at large, and which transmit information to third-party service provider(s) 155 (e.g., YouTube, Facebook, etc.) and/or an AME 145 via the network 125.

User(s) 135 include any individuals who access media content on one or more user device(s) 130. In some examples, the user(s) 135 can be AME panelist(s). For example, the user(s) 135 may be part of an AME-based panel and agree to have their user media device content consumption monitored. In such examples, the occurrence of access and/or exposure to media creates a media impression (e.g., viewing of an advertisement, a movie, a web page banner, a webpage, etc.). Also, in some examples, the user(s) 135 include panelists that have provided their demographic information when registering with the example AME 145. When the example user(s) 135 who are panelists utilize example user device(s) 130 to access media content through the example network 125, the AME 145 (e.g., AME servers) stores panelist activity data associated with their demographic information in the example panel database 150. Also, in some examples, the user(s) 135 can include any individuals who are not panelists (e.g., not registered with the AME 145). Additionally or alternatively, the user(s) 135 can include individuals who are subscribers to services provided by the third-party service provider(s) 155 and utilize these services via their user device(s) 130.

Care provider(s) 140 can include a mental health professional, a home-based caretaker, and/or a family member serving as a caretaker for the patient. Care provider(s) 140 can interact with the patient monitoring system 165 by providing information regarding the patient's well-being that can be included in the behavioral data 170 and/or the clinical data 175. Data provided by the care provider(s) 140 can include, for example, any additional symptoms that are not already of record in the clinical data 175. In some examples, the care provider(s) 140 can also set specific triggers, through the example patient monitoring system 165, that determine what types of behaviors and/or physiological data and/or environmental data can be construed as putting at patient(s) 105 at a high risk (e.g., high risk of self-harm or high-level of deviation from normal behavior). For example, the care provider(s) 160 can identify specific search terms, media content, and/or websites that would trigger an alert to the patient care network (e.g., a patient at a high risk of self-harm searching for keywords that would be associated with self-harm intentions). In some examples, the care provider(s) 140 can designate specific locations that could endanger the patient (e.g., isolated locations, bodies of water, high-traffic areas, etc.). In some examples, the patient monitoring system 165 can communicate with the patient wearable device(s) 115 to determine when such triggers are met and subsequently alert the care provider(s) of the specific behaviors, physiological data, and/or environmental data that are identified as posing a potential hazard for the patient.

The AME 145 operates as an independent party to measure and/or verify audience measurement information relating to media accessed by user(s) 135 of, for example, third-party service provider(s) 155. For example, the AME 145 can store panel meter data in the example panel database 150, the data including impression durations (e.g., length of time that a webpage was viewed by a user, on user device(s) 130). For user(s) 135 who are also AME panelists, the AME 145 can identify user(s) 135 behavior (e.g., web content accessed, popular search terms, length of time that specific media content is accessed for, etc.) based on demographic information provided at the time that the user(s) 135 register with the AME 145. For example, the data collected by the AME 145 can be categorized based on demographics (e.g., females 15-20 years old, males 15-20 years old, females 21-26 years old, males 21-26 years old, etc.). The AME 145 can provide this information to the patient monitoring system 165 to allow the patient monitoring system 165 to establish a population behavioral baseline for specific patient demographic groups.

Panel database 150 can include meter data obtained by the AME 145 for user(s) 135 who are also AME 145 panelists when using user device(s) 130 to view and/or search for media content. The panel database 150 can include meter data collected from various meters (e.g., a people meter, etc.) that are used as audience measurement tools to measure viewing habits of, for example, television and cable audiences (e.g., user(s) 135). Panel meter database 150 can include, for example, demographic information of the media viewer (e.g., user(s) 135 who are AME 145 panelists) and their viewing status (e.g., media content being watched by the panelist(s)). In the example of FIG. 1, the panel database 150 can be used by the patient monitoring system 165 to establish a population behavioral baseline for a specific demographic that relates to a patient 105 demographic. The population behavioral baseline is compared to the patient behavioral data.

Third-party service provider(s) 155 can be any service provider(s) (e.g., a cable company, YouTube, Facebook, Twitter, etc.) that provide media content to user(s) 135 (e.g., on a television, on a website, through an app, etc.) via user device(s) 130. In some examples, the AME 145 can partner with the third-party service provider(s) 155 to collect census-level information that identifies access of media content by user(s) 135 in terms of the total number of times a media content is accessed and the duration of the media content views, without providing specific demographic information (e.g., if the user(s) 135 are third-party service provider 155 subscribers but are not AME 145 panelists). In some examples, the patient monitoring system 165 can have direct access to the media content viewing information of user(s) 135 who are third-party service provider 155 subscribers and/or AME 145 panelists. The third-party service provider(s) 155 can store the collected data concerning the media content usage statistics of user(s) 135 in the example provider database 160.

Provider database 160 includes information that can be stored by the third-party service provider(s) 155 relating to media usage statistics of user(s) 135 who are third-party service provider 155 subscribers. Media usage statistics may include the total number of times a media content was accessed (e.g., number of times a movie was viewed or a webpage was accessed), as well as the duration of the media content access. In some examples, the third-party service provider(s) 155 can access the provider database 160 in order to share information with the AME 145 if the AME 145 has partnered with the third-party service provider(s) 155 to retrieve census-level information (e.g., census-level audience size data and/or census-level impression duration data). In some examples, the provider database 160 can include media content viewing information for user(s) 135 who are both AME 145 panelists and subscribers to services provided by the third-party service provider(s) 155.

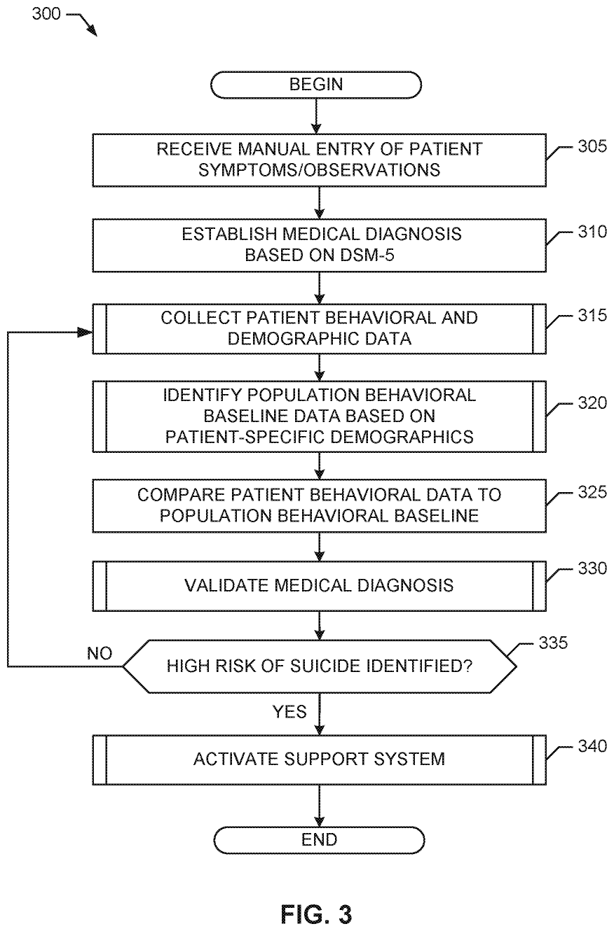

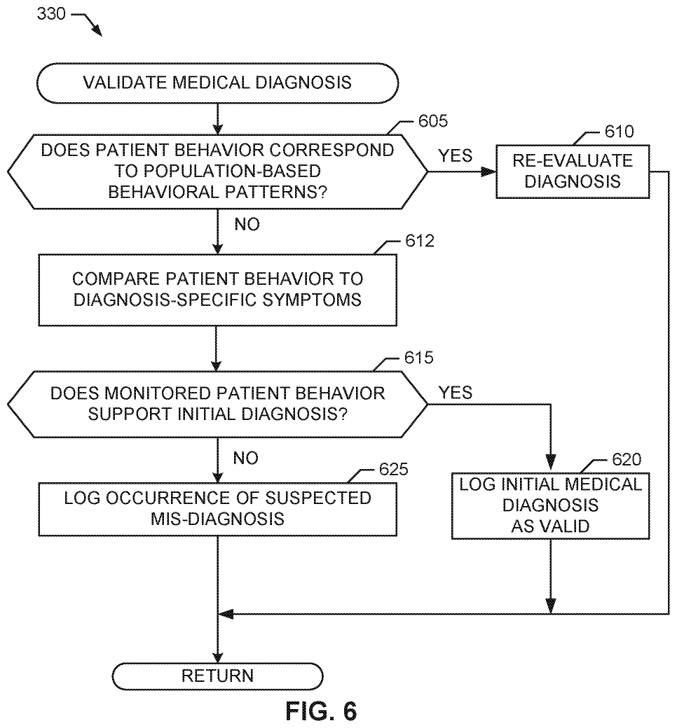

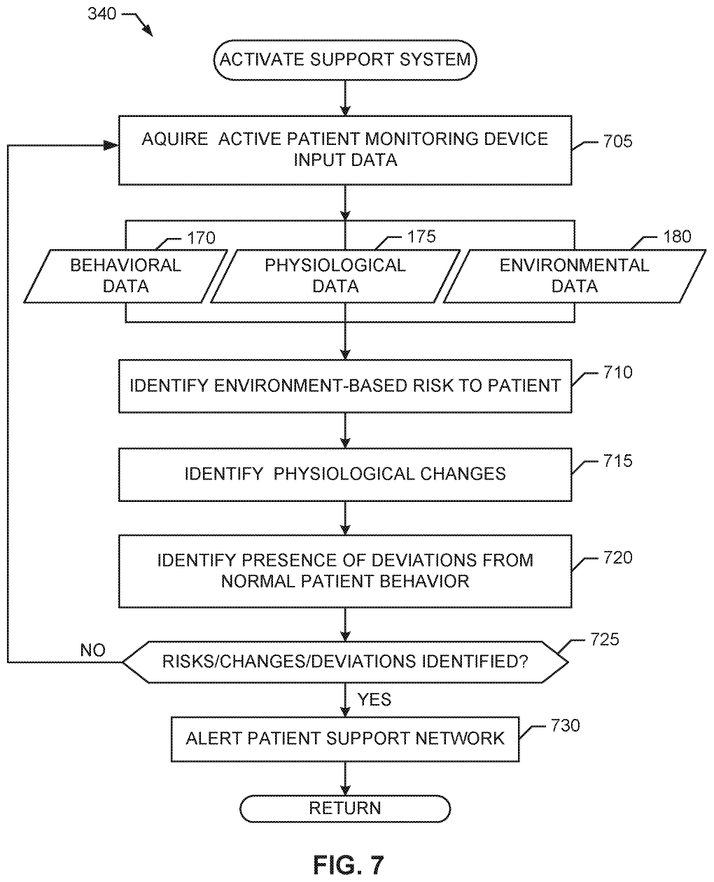

Patient monitoring system 165 is used to determine patient(s) 105 behavior(s) based on a number of collected data points, including data collected by the diagnosis assistant 110, the patient wearable device(s) 115, the real-time social interactor 120, the care provider(s) 140, the AME 145, and/or the third-party service provider(s) 155. The patient monitoring system 165 is used to validate a mental illness diagnosis established by mental health professional(s) and assist in monitoring patient(s) 105. In some examples, the patient monitoring system 165 receives an entry of the patient(s) 105 symptoms and observations of patient(s) 105 behavior from the care provider(s) 140. For example, the care provider(s) 140 may manually enter patient symptoms and behavior data to the monitoring system 165 or via a network 125. In some examples, the entry can include a mental illness diagnosis based on the DSM-5. In some examples, the diagnosis is provided via the diagnosis assistant 110. In some examples, to validate a mental illness diagnosis, the patient monitoring system 165 collects patient behavioral and demographic data via the patient wearable device(s) 115 in order to establish a behavioral baseline for the patient(s) 105 and to compare the baseline to a population behavioral baseline (e.g., derived from user(s) 135 who are subscribers and/or panelists) based on the demographic category of the patient(s) 105, as described in connection with FIGS. 3-5. In some examples, the patient monitoring system 165 validates the medical diagnosis based on whether patient behavior corresponds to population-based behavioral patterns, as described in connection with FIG. 6. In some examples, the patient monitoring system 165 determines the level of risk associated with the patient behavior (e.g., a risk indicative of a suicide attempt by the patient and/or intentions of self-harm). In some examples, the patient monitoring system 165 activates a support system to monitor patient behavior, including environmental data and/or physiological data. In some examples, care provider(s) 140 can set triggers using the patient monitoring system 165 to receive an alert and/or notification when one or more patient-based symptoms are indicative of a risk of self-harm, as described in connection with FIG. 7. In some examples, the patient monitoring system 165 receives data from a real-time social interactor 120 as part of patient-based assessment.

Behavioral data 170 can include any data related to patient(s) 105 behavior(s) (e.g., device usage, media exposure, etc.) that can be used to establish a behavioral baseline for the patient. In some examples, the behavioral data 170 can include population-based behaviors based on data received by the patient monitoring system 165 from the AME 145 and/or the third-party service provider(s) 155, such that the population-based behaviors (e.g., device(s) 130 usage, media exposure of user(s) 135, etc.) can be used to establish the population behavioral baseline. In some examples, the behavioral data 170 can include information provided by care provider(s) 140 to the patient monitoring system 165, data collected from patient wearable device(s) 115, and/or data collected from the diagnosis assistant 110.

Clinical data 175 can include data provided by the care provider(s) 140 and/or collected when establishing a medical diagnosis related to patient(s) 105 mental health (e.g., provided by the diagnosis assistant 110). In some examples, the clinical data includes physiological data collected when monitoring patient(s) 105 (e.g., after activation of a support system) to determine physiological data including, for example, heart rate, blood pressure, sleep pattern, etc.

Environmental data 180 can include data related to the patient(s) 105 environment such as, for example, a location, sound(s) associated with the location (e.g., car horns, train horns, etc.), smell(s) associated with the location (e.g., smoke), ambient temperature, lighting, movement indicative of being in a vehicle, chemical information (e.g., levels of carbon monoxide), etc.). In some examples, the patient monitoring system 165 collects the environmental data 180 via the patient wearable device(s) 115. For example, the patient monitoring system 165 may collect environmental data 180 when a support system is engaged to monitor any risks to the patient (e.g., risk of self-harm). In some examples, the environmental data 180 can be used to notify and/or alert care provider(s) 105 when a patient 105 is located in an area deemed to be of potential risk to the patient (e.g., an isolated area, a railroad crossing, an elevated bridge, etc.). In some examples, the environmental data can also be provided based on data collection from surveillance cameras. For example, a patient identified to be at a high risk of suicide may additionally be monitored to determine the patient's presence in locations known to have a history of suicide attempts and/or fatalities (e.g., building rooftops, railroad crossings, railroad stations, bridges, etc.). In some examples, surveillance cameras can be positioned in such high-risk locations based on available data on location-based death statistics and/or suicide attempts.

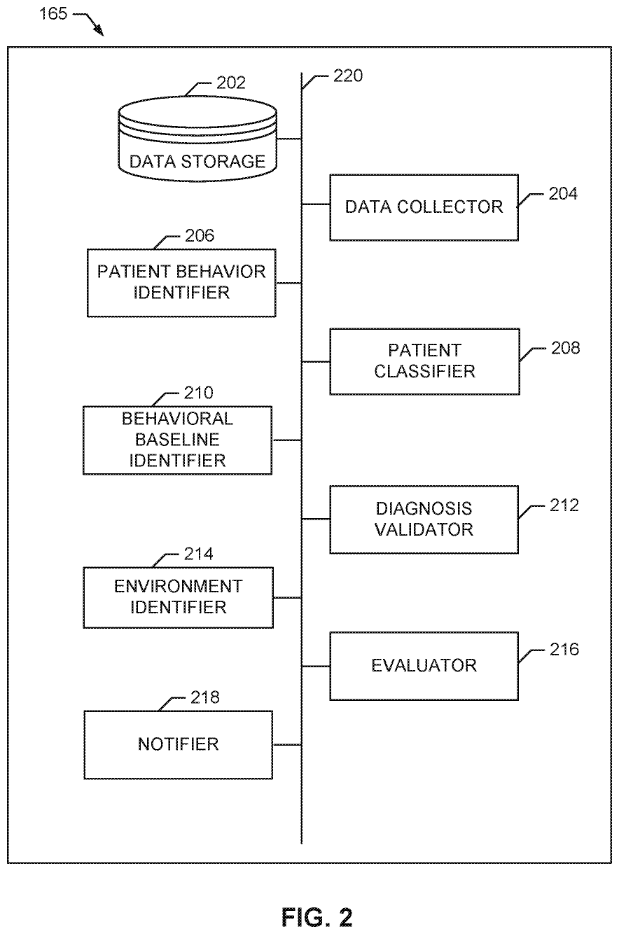

FIG. 2 is a block diagram of an example implementation of the patient monitoring system 165. The patient monitoring system 165 includes example data storage 202, an example data collector 204, an example patient behavior identifier 206, an example patient classifier 208, an example behavioral baseline identifier 210, an example diagnosis validator 212, an example environment identifier 214, an example evaluator 216, and an example notifier 218, which are connected using an example bus 220.

The data storage 202 stores data associated with patient(s) 105 that is used to determine a behavioral baseline for the patient(s) 105. In some examples, the data stored in the data storage 202 can include the behavioral data 170, the clinical data 175, and/or the environmental data 180. For example, when a mental illness diagnosis is validated, the data storage 202 stores patient(s) 105 behavioral data in order for the patient monitoring system 165 to establish a patient-based behavioral baseline. In some examples, the data storage 202 includes data related to the user(s) 135 to determine a population behavioral baseline. For example, the data storage 202 can include data retrieved from the third-party service provider(s) 155 (e.g., from the provider database 160) and/or data retrieved from the AME 145 (e.g., from the panel database 150). In some examples, the data storage 202 includes data received by the patient monitoring system 165 from one or more of the patient wearable device(s) 115, the diagnosis assistant 110, the real-time social interactor 120, the care provider(s) 140, the AME 145, and/or the third-party service provider(s) 155. In some examples, the data storage 202 includes patient-specific demographic data (e.g., female/male, age, etc.). In some examples, the patient-specific demographic data includes data from an AME panel meter (e.g., data from the panel database 150). The data storage 202 may be implemented by any storage device and/or storage disc for storing data such as, for example, flash memory, magnetic media, optical media, etc. Furthermore, the data stored in the data storage 202 may be in any data format such as, for example, binary data, comma delimited data, tab delimited data, structured query language (SQL) structures, etc. While in the illustrated example the data storage 202 is illustrated as a single database, the data storage 202 can be implemented by any number and/or type(s) of databases.

The data collector 204 collects patient(s) 105 behavioral data during a patient assessment period to establish a patient behavioral baseline. In some examples, the data collector 204 collects data that is relevant to establishing a patient behavioral baseline once a health care professional evaluates the patient 105 and determines a presence of a mental illness. The patient behavior data can include data relating to media exposure, patient device usage, etc. Such data can be any data that is also collected for the general population (e.g., non-panelist and panelist user(s) 135) from the third-party service provider(s) 155 and/or the AME 145. In some examples, the data collector 204 retrieves the data from patient wearable device(s) 115, including any devices used by the patient on a regular basis for media content retrieval and/or Internet usage, etc. (e.g., a television, a smartphone, etc.). In some examples, the data collector 204 retrieves data from the AME 145 if the patient is also a panelist of the AME 145 (e.g., the data collected from patient devices using a panel meter, set top box meter, etc.). For example, the data collector 204 collects data of patient(s) 105 device usage to determine, for example, keywords searched, media exposure (e.g., movies and/or videos watched, websites visited, etc.). In some examples, the patient(s) 115 can be AME 145 panelist(s), such that the AME 145 collects data on impression (e.g., media exposure), including impression counts (e.g., number of times media exposure occurred), and/or impression count durations (e.g., length of time the user was exposed to the media). In some examples, the data collector 204 supplements the data retrieved from the patient wearable device(s) 115 using data from the care provider(s) 140.

The patient behavior identifier 206 identifies a patient-specific behavioral baseline for the patient(s) 105. In some examples, the patient behavior identifier 206 identifies a patient-specific behavioral baseline based on the data collected using the data collector 204 from a patient-based assessment performed by a mental health professional. In some examples, the patient behavior identifier 206 uses data collected from one or more patient devices, including patient wearable device(s) 115 and/or patient computing devices (e.g., a smartphone, etc.) and/or any device(s) that provide media exposure statistics (e.g., via a panel meter if the patient is an AME 145 panelist). In some examples, the patient behavior identifier 206 uses data collected by the data collector 204 to determine how often a patient engages in certain activities (e.g., searching for specific keywords, exposure to a specific movie genre, etc.). In some examples, the patient behavior identifier 206 determines a patient behavior baseline based on the frequency of such behaviors, in combination with any other data accessible via the data collector 204. For example, the patient behavior identifier 206 can establish frequency of a patient's use of social media and/or usage of specific blogging sites and/or frequency of using certain keywords when search for media content or posting comments on a blog. For example, a patient diagnosed with Attention Deficit/Hyperactivity Disorder (ADHD) typically displays symptoms that can include trouble paying attention, sitting still, or finishing tasks. Such a patient is likely to show similar behaviors when using media devices (e.g., starting multiple videos without viewing them to completion, etc.). However, media usage-based behavior and media exposure can differ based on patient demographics (e.g., online behavior of teenagers can vary from that of older adults).

Establishing a mental illness diagnosis that is accurate can require access to data that can be difficult to obtain from short-term patient observation. In some examples, the patient behavior identifier 206 establishes a behavioral baseline based on multiple sources. For example, the patient behavior identifier 206 may validate symptoms based on patient behavior identified from the diagnosis assistant 110, the patient device(s) 115, and/or the care provider(s) 140. For example, by establishing a behavioral baseline for the patient(s) 105 based on media exposure (e.g., statistics retrieved from AME 145 and/or third party service provider(s) 155) using the patient behavior identifier 206, the patient monitoring system 165 can compare the patient behavioral baseline to a population behavioral baseline determined by the behavioral baseline identifier 210.

The patient classifier 208 classifies a patient into a demographic category based on patient data. For example, the patient classier 208 determines patient-specific information as it relates to demographics (e.g., female/male, age, etc.). The patient classifier 208 can classify the patient into a demographic category (e.g., female, 20-25 years of age). In some examples, the patient classifier 208 classifies the patient based on the population-level (e.g., user(s) 135) data available from the third-party service provider(s) 155 and/or the AME 145.

The behavioral baseline identifier 210 identifies a population behavioral baseline based on a patient-specific demographic data. For example, behavioral baseline identifier 210 can access patient-specific demographic data from the patient classifier 208 to determine the demographic to use when retrieving population-based data (e.g., data from panelist and non-panelist user(s) 135). In some examples, behavioral baseline identifier 210 determines the population behavioral baseline as it relates to the user(s) 135 media exposure and device(s) 130 usage. For example, the behavioral baseline identifier 210 accesses the panel database 150 and/or the provider database 160 to retrieve information on panelist and/or non-panelist user(s) 135 that is specific to the patient-based demographic classification. For example, if the patient is male and between the ages of 20-25, the behavioral baseline identifier 210 identifies a population behavioral baseline that is specific to user(s) 135 within that demographic category. For example, the data can be specific to the user(s) 135 media exposure (e.g., impressions, impression counts, and/or impression durations). In some examples, the data may be specific to a third-party service or content provider. For example, if the patient(s) 105 are users of Facebook and/or YouTube, the behavioral baseline identifier 210 can retrieve data on user(s) 135 specific to the use of these service or content providers. As such, the behavioral baseline identifier 210 determines a population-specific behavioral baseline that may take into account user(s) 135 online behavior and/or media exposure. For example, if a patient 105 baseline behavior as identified by the patient behavior identifier 206 indicates that the patient 105 frequently and/or regularly watches a specific movie genre or uses certain key words (e.g., “depressed”, “sad”, “anxious”, “angry”, etc.) on a frequent and/or regular basis, this behavior can be compared to that of the population behavioral baseline that is based on the patient-specific demographic category to establish whether the patient behavior deviates from that of the population-based behavior. For example, patients who are young adults can be more likely to use certain key words in an online setting as compared to patients who are older individuals. In some examples, the demographics used to establish the behavioral baseline can include other accessible demographic information (e.g., educational level, occupation, income, etc.).

The diagnosis validator 212 identifies the diagnosis as valid based on whether patient behavior corresponds to population-based behavioral patterns. For example, the diagnosis validator 212 determines whether there is deviation of the patient-based behavioral baseline from the population-specific behavioral baseline. For example, if the patient behavior corresponds to population-based behavioral patterns (e.g., a high correlation), the diagnosis validator 212 considers whether the monitored patient behavior supports the initial diagnosis. For example, the diagnosis validator 212 can determine the behavioral patterns that can be expected from patients with a specific mental illness diagnosis and compare these to the patient(s) 105 behaviors. For example, if a patient is diagnosed with a bipolar disorder (e.g., condition marked by alternating periods of elation and depression), certain physical symptoms (e.g., mood swings, emotional highs and lows, etc.) can also transfer to behaviors that are expressed through media usage (e.g., comments that use contrasting words expressing the emotional state of the user, variance in media exposure preferences, etc.). As such, some behavioral patterns can be more likely to be observed in patients with a bipolar disorder that may not be observed in patients who suffer from depression only. The diagnosis validator 212 determines whether the monitored patient behavior supports the initial diagnosis by determining whether there is a low correlation with population-based behavior or a high correlation with population-based behavior. Based on the determination of the correlation of patient behavior with population-based behavior, the diagnosis validator 212 assesses the validity of the diagnoses. In some examples, the diagnosis validator 212 logs occurrence of a suspected mis-diagnosis if the monitored patient behavior does not support the initial diagnosis. In some examples, if the diagnosis validator 212 determines that the monitored patient behavior supports the initial diagnosis, the diagnosis validator 212 logs the initial diagnosis as valid.

The environment identifier 214 determines the patient-based environment based on data collected from one or more patient wearable device(s) 115. For example, the environment identifier 214 can be used to monitor patient(s) 105 who are identified by the patient monitoring system 165 to be at high risk (e.g., of self-harm) based on the process of validating the patient's medical diagnosis. For example, if patient behavior that is indicative of an intent to do self-harm (e.g., keyword searches including “suicide”, etc.) is discovered during the establishment of the patient behavioral baseline, the patient monitoring system 165 can activate a support system that provides additional patient monitoring to assess for certain triggers (e.g., patient location) that can be used to inform the care provider(s) 140 in the event that a patient is perceived to be in danger. As such, the patient monitoring system 165 uses the environment identifier 214 to determine the patient's locations and/or other location-specific features of the given environment (e.g., noises, smells, chemical exposures, etc.).

The evaluator 216 compares the patient behavioral baseline to the population behavioral baseline to determine a correlation between the patient baseline and the population baseline during patient monitoring. For example, once a diagnosis has been validated, the patient can be monitored to continue to determine whether interventional procedures (e.g., cognitive behavioral therapy, etc.) are effective in reducing symptoms and/or behaviors that are used to validate the medical diagnosis. For example, as the population behavioral baseline can change over time, the patient behavioral baseline is also expected to adjust over time during the course of treatment. As such, the evaluator 216 can be used to monitor the correlation between the patient baseline and the population behavioral baseline over time. In some examples, the evaluator 216 can be used by the diagnosis validator 212 to perform another diagnosis validation 212 as needed based on the comparison of the population behavioral baseline to the patient baseline.

The notifier 218 alerts a patient support network when a safety risk to a patient is identified, the safety risk including an environment-based risk, a physiological change, and/or a deviation from the patient behavioral baseline. For example, the patient monitoring system 165 activate a support system when the patient is identified as being at risk (e.g., of self-harm). In such examples, the patient monitoring system 165 can receive additional input from the real-time social interactor 120, in addition to the patient wearable device(s) 115. In some examples, the risk level of the patient behavior includes a risk indicative of a suicide attempt by the patient. The activated support system collects patient behavioral data 170, patient physiological data 175, and/or patient-specific environmental data 180. The notifier 218 can alert the care provider(s) 140 when the patient is perceived to be at a high risk or is engaging in a behavior that puts the patient at risk (e.g., determined using the environment identifier 214).

While an example manner of implementing the patient monitoring system 165 is illustrated in FIGS. 1 and 2, one or more of the elements, processes and/or devices illustrated in FIGS. 1 and 2 may be combined, divided, re-arranged, omitted, eliminated and/or implemented in any other way. Further, the example data storage 202, the example data collector 204, the example patient behavior identifier 206, the example patient classifier 208, the example behavioral baseline identifier 210, the example diagnosis validator 212, the example environment identifier 214, the example evaluator 216, the example notifier 218 and/or, more generally, the example patient monitoring system 165 of FIGS. 1-2 and/or the example patient wearable device(s) 115, the example diagnosis assistant 110, the example real-time social interactor 120, and/or the example user device(s) 130 of FIG. 1 may be implemented by hardware, software, firmware and/or any combination of hardware, software and/or firmware. Thus, for example, any of the example data storage 202, the example data collector 204, the example patient behavior identifier 206, the example patient classifier 208, the example behavioral baseline identifier 210, the example diagnosis validator 212, the example environment identifier 214, the example evaluator 216, the example notifier 218, and/or, more generally, the example patient monitoring system 165 of FIGS. 1-2 and/or the example patient wearable device(s) 115, the example diagnosis assistant 110, the example real-time social interactor 120, and/or the example user device(s) 130 of FIG. 1 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example data storage 202, the example data collector 204, the example patient behavior identifier 206, the example patient classifier 208, the example behavioral baseline identifier 210, the example diagnosis validator 212, the example environment identifier 214, the example evaluator 216, and/or the example notifier 218, the example patient wearable device(s) 115, the example diagnosis assistant 110, the example real-time social interactor 120, the example user device(s) 130, and/or the example patient monitoring system 165 is/are hereby expressly defined to include a non-transitory computer readable storage device or storage disk such as a memory, a digital versatile disk (DVD), a compact disk (CD), a Blu-ray disk, etc. including the software and/or firmware. Further still, the example patient wearable device(s) 115, the example diagnosis assistant 110, the example real-time social interactor 120, the example user device(s) 130, and/or the example patient monitoring system 165 may include one or more elements, processes and/or devices in addition to, or instead of, those illustrated in FIGS. 1 and 2, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

Flowcharts representative of example machine readable instructions for implementing the example patient monitoring system 165 of FIGS. 1-2 are shown in FIGS. 3-7, respectively. The machine-readable instructions may be one or more executable programs or portion(s) of an executable program for execution by a processor such as the processor 812 shown in the example processor platform 800 discussed below in connection with FIGS. 3-7. The program may be embodied in software stored on a non-transitory computer readable storage medium such as a CD-ROM, a floppy disk, a hard drive, a digital versatile disk (DVD), a Blu-ray disk, or a memory associated with the processor 812, but the entire program and/or parts thereof could alternatively be executed by a device other than the processor 812 and/or embodied in firmware or dedicated hardware. Further, although the example program is described with reference to the flowcharts illustrated in FIGS. 3-7, many other methods of implementing the example patient monitoring system 165 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, etc. in order to make them directly readable and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example processes of FIGS. 3, 4, 5, 6 and/or 7 may be implemented using executable instructions (e.g., computer and/or machine readable instructions) stored on a non-transitory computer and/or machine readable medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term non-transitory computer readable storage medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

FIG. 3 is a flowchart 300 representative of machine readable instructions that may be executed to implement elements of the example patient monitoring system 165 of FIGS. 1-2. With reference to the preceding figures and associated written descriptions, the example program 300 of FIG. 3 begins execution with the patient monitoring system 165 receiving manual entry of patient symptoms and/or observations (block 305). For example, the patient monitoring system 305 receives the patient symptoms and/or observation from the care provider(s) 140 via the network 125. In some examples, the patient monitoring system 165 receives the patient-related medical diagnosis via the diagnosis assistant 110, which can be used to determine the medical diagnosis based on the DSM-5 (block 310).

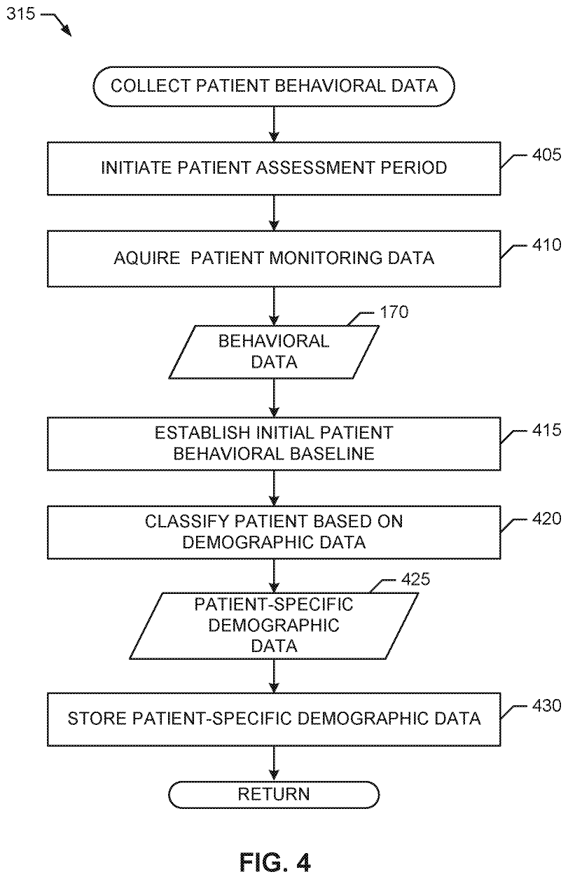

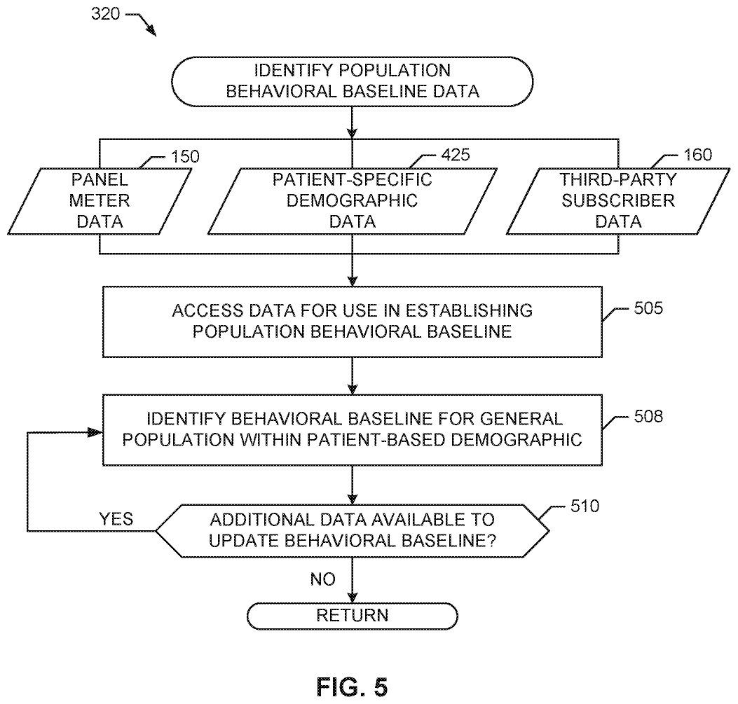

The patient monitoring system 165 collects patient behavioral and demographic data to establish a patient behavioral baseline (block 315), as detailed in connection with FIG. 4. For example, the data collector 204 receives data from patient wearable device(s) 115 and/or any other patient-specific device (e.g., a smartphone, a television, etc.) that the patient uses for media exposure and consumption. In some examples, the data collection includes patient-specific data from the third-party service provider(s) 155 and/or the AME 145 (e.g., data retrieved from the provider data base 160 and/or the panel database 150), which is used by the patient behavior identifier 206 to identify an initial patient behavioral baseline. In some examples, the patient classifier 208 classifies the patient into a specific demographic category (e.g., based on gender, age, etc.). Once the patient-specific demographics are identified, the behavioral baseline identifier 210 identifies the population behavioral baseline based on the patient-specific demographics (block 320), as detailed in connection with FIG. 5.

The diagnosis validator 212 compares the patient behavioral data to the population behavioral baseline (block 325). In addition, the diagnosis validator 212 uses the results of the comparison to validate the medical diagnosis (block 330), as detailed in connection with FIG. 6. In some examples, the diagnosis validator 212 determines whether the patient is at risk (e.g., a high risk of suicide or self-harm intentions) (block 335). If the diagnosis validator 212 determines that a risk level is low (e.g., the patient does not require monitoring and is not at risk of self-harm), the patient monitoring system 165 continues to monitor the patient by collecting behavioral data to ensure that there are no additional or subsequent deviations from the established behavioral baseline. In some examples, if the diagnosis validator 212 determines that the patient is at high risk of suicide or other self-harm (block 335), the patient monitoring system 165 activates a support system to provide additional options for monitoring the patient (block 340), as detailed in connection with FIG. 7.

FIG. 4 is a flowchart representative of machine readable instructions that may be executed to implement elements of the example patient monitoring system 165 of FIGS. 1-2. The flowchart of FIG. 4 is representative of instructions used to collect patient behavioral data. Once the patient monitoring system 165 receives an initial mental illness diagnosis, the patient monitoring system 165 initiates a patient assessment period to collect patient behavioral data (block 405). The data collector 204 acquires patient monitoring data from patient 105 devices, including patient wearable device(s) 115 that can include patient-based media exposure activities (block 410). In some examples, the behavioral data 170 received by the data collector 204 can include any data associated with patient 105 device usage (e.g., information obtained from the third-party service provider(s) 155 and/or the AME 145).

The patient behavior identifier 206 establishes an initial patient behavioral baseline based on the collected patient behavioral data (block 415). For example, the patient behavioral baseline can be associated with the frequency of patient(s) 105 exposure to certain media and/or internet activities (e.g., frequency of specific keyword usage, websites of interest, length of media exposure, times of media exposure, etc.). The patient classifier 208 classifies the patient into a specific demographic category based on demographic data provided by the patient and/or care provider(s) 140 (block 420). The patient monitoring system 165 stores patient-specific demographic data 425 for example in the data storage 202 (block 430). As disclosed herein, the patient-specific demographic data is used to determine the population behavioral baseline that is based on the patient demographic categorization. For example, the patient-specific demographic data stored in the data storage can be used by the patient monitoring system 165 as input to the behavioral baseline identifier 210 in combination with third-party service provider data 160 and/or AME panel meter data 150.

FIG. 5 is a flowchart representative of machine readable instructions that may be executed to implement elements of the example patient monitoring system 165 of FIGS. 1-2. The flowchart of FIG. 5 is representative of instructions used to identify population behavioral data. The behavioral baseline identifier 210 identifies the population behavioral baseline for use by the patient monitoring system 165 as a comparison to the patient behavioral baseline as part of validating the medical diagnosis. In some examples, the behavioral baseline identifier 210 accesses data that is used to establish the population behavioral baseline (block 505). For example, the behavioral baseline identifier 210 accesses data including the panel meter data 150 from the AME 145, the third-party subscriber data 160 from the third-party service provider(s) 155, and/or the patient-specific demographic data 425 used by the patient classifier 208 to classify the patient into a specific demographic category.

The behavioral baseline identifier 210 identifies a behavioral baseline for the general population within the patient-based demographic category (block 508). For example, the behavioral baseline identifier 210 analyzes the data associated with panelist and non-panelist user(s) 135 to determine, for example, how user(s) 135 media usage statistics compare to that of the patient(s) 105. In some example, the media exposure data can include impressions (e.g., videos viewed), impression counts (e.g., number of times webpage accessed), and/or impression duration (e.g., length of time media exposure occurs and at what times the exposure occurs). In some examples, the analysis includes a comparison of keywords used by the user(s) 135 (e.g., number of times the user(s) 135 use certain words to describe their feelings/opinions, etc.).

Over time, the behavioral baseline can be updated when additional data becomes available, allowing the baseline to be adjusted based on cumulative changes in the user(s) 135 behavior (e.g., movie genre preferences for given demographic, web site usage, and/or type of audio content accessed). For example, the behavioral baseline identifier 210 uses and/or works in concert with the data collector 204 to determine if additional data is available to update the behavioral baseline (block 510). If the behavioral baseline identifier 210 determines that additional data is available to update the behavioral baseline (block 515), the behavioral baseline identifier 210 accesses data that is used to establish the population behavioral baseline (block 505) and the program continues with the updated data. If the behavioral baseline identifier 210 determines that there is no additional data available to update the behavioral baseline (block 510), the patient monitoring system 165 proceeds to compare the patient behavioral data to the population behavioral baseline determined using the behavioral baseline identifier 210.

FIG. 6 is a flowchart representative of machine readable instructions that may be executed to implement elements of the example patient monitoring system 165 of FIGS. 1-2. The flowchart of FIG. 6 is representative of instructions used to validate a medical diagnosis. Once the behavioral baseline identifier 210 establishes the population behavioral baseline, the diagnosis validator 212 validates the medical diagnosis. For example, the diagnosis validator 212 determines whether patient behavior corresponds to population-based behavioral patterns (block 605). In some examples, the behavioral patterns can include type of media exposure, duration of media exposure, keywords used during online searches, types of keywords used to communicate with others on social sites, sites of interest, etc. In some examples, if the diagnosis validator 212 determines that the patient behavior corresponds to population-based behavioral patterns (block 605), the diagnosis validator 212 can initiate a re-evaluation of the medical diagnosis (block 610). In some examples, an evaluator 216 can be used to determine the level of correlation (e.g., a high correlation, a low correlation) between the patient behavioral baseline and population behavioral baseline. In some examples, a high correlation between the two baselines can indicate that the patient behavior does correspond to population-based behavior that is established based on the determined patient demographic category. In some examples, patient behavior corresponding to population-based behavior is indicative that the patient behavior is normal and, therefore, should not cause alarm or indicate the patient is at risk of self-harm or suicide. Thus, in such examples, the diagnosis validator 212 can initiate a re-evaluation of the medical diagnosis (block 610).

If the diagnosis validator 212 determines that the patient behavior does not correspond to population-based behavioral patterns (block 605), the diagnosis validator 212 compares patient behavior to diagnosis-specific symptoms that would be expected in the observed patient behavior if the initial diagnosis is valid (block 612). For example, the patient monitoring system 165 receives the initial diagnosis via the diagnosis assistant 110 (e.g., based on the DSM-5) and/or the care provider(s) 140. The diagnosis validator 212 can use the patient behavioral data 170, collected by the data collector 204, to compare the behavioral data of the patient against the behavior(s) and/or symptoms that are expected when a patient is diagnosed with a specific mental health condition. In some examples, the diagnosis validator 212 determines that the monitored patient behavior does support the initial diagnosis (block 615), resulting in the diagnosis validator 212 logging the initial medical diagnosis as a valid diagnosis (block 620). In some examples, the diagnosis validator 212 determines that the monitored patient behavior does not support the initial diagnosis (block 615), in which case the diagnosis validator 212 logs the occurrence of a suspected mis-diagnosis (block 625). For example, if the behavioral data collected by the data collector 204 supports the diagnosis (e.g., a patient with a bipolar diagnosis with behavioral data showing indications of drastic mood changes as expressed in the way that the patient communicates over a prolonged period of time), the diagnosis can be validated. For example, if the behavioral data collected by the data collector 204 does not support the diagnosis (e.g., a patient with a diagnosis of severe depression presents with behavioral data showing indications of periods of elation), the diagnosis may be logged as a potential mis-diagnosis.

FIG. 7 is a flowchart representative of machine readable instructions that may be executed to implement elements of the example patient monitoring system 165 of FIGS. 1-2. The flowchart of FIG. 7 is representative of instructions used to activate a support system. Once the diagnosis validator 212 has identified the initial mental illness diagnosis as a valid diagnosis or a mis-diagnosis, the patient monitoring system 165 can determine whether the patient is in need of additional monitoring. For example, the patient monitoring system 165 can determine whether the patient is at risk of engaging in behaviors that can be harmful (e.g., a risk of self-harm). For example, once the diagnosis validator 212 has established that the patient has a valid diagnosis of severe depression, the patient monitoring system 165 can activate a support system to continue patient-based monitoring for any behaviors that can be identified as presenting a risk to the patient. For example, the patient monitoring system 165 acquires active patient monitoring device input data (block 705) via the real-time social interactor 120 (e.g., a robot, smartphone application, etc.). The real-time social interactor 120 can communicate with the patient and/or solicit patient feedback to determine the patient's mental state and assist in determining patient behavior, as well as to monitor whether interventional therapies are effective. The patient monitoring system 165 can continue to acquire data that would be necessary to determine whether there is any risk to the patient (e.g., behavioral data 170, physiological data 175, and/or environmental data 180). For example, such data can be collected from one or more of the patient wearable device(s) 115 and/or the real-time social interactor 120.

In some examples, the environmental identifier 214 can be used to determine whether a risk to the patient exists based on the patient-specific environment (e.g., location, sounds, etc.) (block 710). In some examples, the care provider(s) 140 can identify specific trigger points to receive an alert from the patient monitoring system 165 when a specific location is identified by the patient monitoring system 165 via the patient wearable device(s) 115. In some examples, the patient monitoring system 165 can identify physiological changes that deviate from normal (e.g., heart rate, blood pressure, sleep patterns, etc.). In some examples, the patient monitoring system 165 identifies presence of deviations (including, for example, sudden deviations) from normal patient behavior (block 720) based on the behavioral data 170, physiological data 175, and/or environmental data 180.

In some examples, the evaluator 216 can be used to revise the patient behavioral baseline over time and compare to any updated population behavioral baselines. If the patient monitoring system 165 identifies any deviations from normal patient behavior and/or any risks to the patient (block 725), the notifier 218 alerts the patient support network (e.g., care provider(s) 140) (block 730). In some examples, the notifier 218 can also generate a notification or alert when a trigger identified by the care taker(s) 140 is present (e.g., specific changes in patient behavioral, physiological, and/or environmental data).

If the patient monitoring system 165 does not identify deviations from normal patient behavior and/or any risks to the patient (block 725), the patient monitoring system 165 continues to acquire active patient monitoring device input data (block 705). For example, the patient monitoring system 165 continues to acquire data from the real-time social interactor 120 and/or patient wearable device(s) 115 until a designated patient monitoring period is complete and/or any interventional therapies are determined to be successful. For example, a therapy may be considered successful when a patient behavioral baseline begins to have a high correlation to a population behavioral baseline, in combination with normalization of any deviating physiological data.

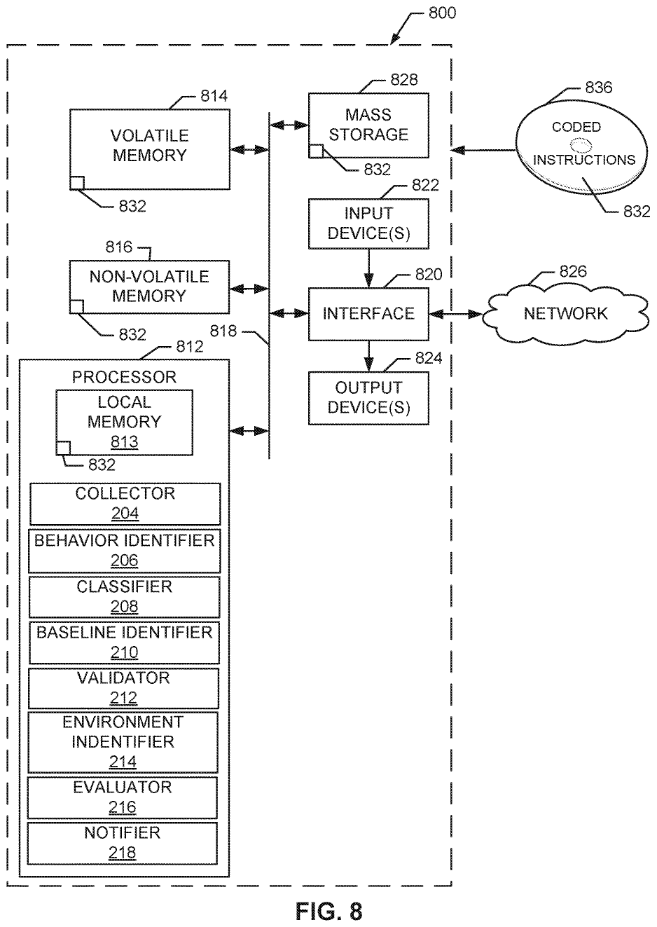

FIG. 8 is a block diagram of an example processing platform structured to execute the instructions of FIGS. 3-7 to implement the example patient monitoring system 165 of FIGS. 1-2. The processor platform 800 may also be used to implement one or more of the diagnosis assistant 110, the patient wearable device(s) 115, the real-time social interactor 120, and/or the user device(s) 130. The processor platform 800 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, or any other type of computing device.

The processor platform 800 of the illustrated example includes a processor 812. The processor 812 of the illustrated example is hardware. For example, the processor 812 can be implemented by one or more integrated circuits, logic circuits, microprocessors, GPUs, DSPs, or controllers from any desired family or manufacturer. The hardware processor 812 may be a semiconductor based (e.g., silicon based) device. In this example, the processor 812 implements the example data collector 204, the example patient behavior identifier 206, the example patient classifier 208, the example behavioral baseline identifier 210, the example diagnosis validator 212, the example environment identifier 214, the example evaluator 216, and the example notifier 218 of FIG. 2.

The processor 812 of the illustrated example includes a local memory 813 (e.g., a cache). The processor 812 of the illustrated example is in communication with a main memory including a volatile memory 814 and a non-volatile memory 816 via a bus 818. The volatile memory 814 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 816 may be implemented by flash memory and/or any other desired type of memory device. Access to the main memory 812, 814 is controlled by a memory controller.

The processor platform 800 of the illustrated example also includes an interface circuit 820. The interface circuit 820 may be implemented by any type of interface standard, such as an Ethernet interface, a universal serial bus (USB), a Bluetooth® interface, a near field communication (NFC) interface, and/or a PCI express interface.

In the illustrated example, one or more input devices 822 are connected to the interface circuit 820. The input device(s) 822 permit(s) a user to enter data and/or commands into the processor 812. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 824 are also connected to the interface circuit 820 of the illustrated example. The output devices 824 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 820 of the illustrated example, thus, typically includes a graphics driver card, a graphics driver chip and/or a graphics driver processor.

The interface circuit 820 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 826. The communication can be via, for example, an Ethernet connection, a digital subscriber line (DSL) connection, a telephone line connection, a coaxial cable system, a satellite system, a line-of-site wireless system, a cellular telephone system, etc.

The processor platform 800 of the illustrated example also includes one or more mass storage devices 828 for storing software and/or data. Examples of such mass storage devices 828 include floppy disk drives, hard drive disks, compact disk drives, Blu-ray disk drives, redundant array of independent disks (RAID) systems, and digital versatile disk (DVD) drives. The mass storage device 828 includes the example data storage 202 of FIG. 2.

Machine executable instructions 832 represented in FIGS. 3-7 may be stored in the mass storage device 828, in the volatile memory 814, in the non-volatile memory 816, and/or on a removable non-transitory computer readable storage medium such as a CD or DVD 836.

From the foregoing, it will be appreciated that example systems, methods, and apparatus permit verification of a mental illness diagnosis based on patient behavioral and demographic data. Furthermore, example technical solutions disclosed herein monitor patients at high risk to establish a comprehensive assessment of their risk of self-harm based on behavioral data, clinical data, and environmental data. Examples disclosed herein leverage a number of techniques for obtaining patient-based information to monitor patients and assess the accuracy of a mental illness diagnosis based on comparison to an established population-derived behavior baseline. Example technical solutions establish behavior baseline of the population according to demographic data based on behavioral data, which can be collected by various third-party service providers (e.g., Facebook, Twitter, etc.), as well as marketing research and/or healthcare research companies. In the examples disclosed herein, panel meter data obtained from an AME (e.g., such as The Nielsen Company (US), LLC) can be leveraged to identify a population behavior baseline in order to compare the population behavior baseline with patient behavioral data as part of validating a mental illness diagnosis. An AME monitors viewing of media presented by media devices, which can be used to extrapolate information relevant to establishing a population and/or patient-based behavior baseline (e.g., demographic-based media content exposure, media content preferences, and/or selections). Examples disclosed herein classify a patient according to the patient's demographic data. The patient behavior is compared with behavior baseline for the same demographics, and the baseline can be updated during data collection from the population at large.

As disclosed herein, in some examples, the patient monitoring system 165 operated in three axles: (1) diagnosis validation, (2) patient monitoring, and (3) therapy support. With respect to the diagnosis validation, the patient monitoring system 165, in some examples, operates in two directions: (a) by validating the patient's own diagnosis by observing if the patient is indeed presenting the behavioral patterns that are expected for the diagnostic made or if they resemble the ones of an alternate diagnosis, and (b) by providing feedback to the diagnostic decision tree itself. Thus, over time, the patient monitoring system 165 is able to identify inconsistencies or weaknesses in the diagnostic model. For example, if one type of mental illness is believed to occur in 20% of the population, and the mental illness is characterized by a pattern of behavior. However, the data acquired from a number of monitored patients and/or from the population at large (e.g., via marketing data and/or other data disclosed herein) may indicate that this pattern of behavior is observed in 70% of the population. In this example, this disparity should trigger an alarm that this pattern of behavior may not be enough evidence alone to determine the diagnosis. In such examples, the patient monitoring system 165, the caregivers, and/or other health care professionals should look for other indicators in the patient's behaviors that would categorize the patient in the 20% group of the population when compared with the population at large. Furthermore, in some examples, the patient monitoring system 165 adds the additional indicators to the decision tree. Additionally or alternatively, the prevalence of the disease (the diagnosis of the disease) among the population should be reviewed.

In some examples, the patient monitoring system 165 makes recommendations for the DSM-5 re-evaluation committee on how diagnosis decision tree can be improved. Also, in some examples, the patient monitoring system 165 provides data depicting the prevalence of misdiagnosis. For example, a patient may fit the criteria for both ADHD and PTSD. However, the patient should be diagnosed primarily with PTSD and treated as such. But a physician may miss the key signs of PTSD and instead start treating the patient as ADHD. In some examples, as disclosed herein, the patient monitoring system 165 may detect the telltale signs or hallmarks of PTSD and alert the treatment team of this fact. The patient monitoring system 165 can log the occurrence of the misdiagnosis. Over time, the patient monitoring system 165 presents data indicative of how frequently there was a misdiagnosis, and this information can also be used to adjust the diagnostic system to make is less prone to this type of mistake.

With regard to the therapy support and the robotic agents, the real-time social interactor 120 including, for example social robot(s) and/or telepresence robot(s), are able to capture behaviors that human observers cannot easily capture. For example, as noted above, patients may answer assessment questions more honestly when the patients are interacting with computers than when interacting with humans because the patients have less fear of self-disclosure and are less afraid of being judged. The intelligence provided in or by the real-time social interactor 120 allows for feedback that can have a positive influence on the patient without the need of human intervention. In addition, the intelligence provided in or by the real-time social interactor 120 can also provide better quality of the data provided to the therapeutic team to make decisions about the patient's treatment.

The examples disclosed herein may be used in a hospital, an assisted living residence, a group residence, a private residence in which the examples disclosed herein interact with more than one patient at a time and observe the interactions among the patients as well. For example, in the examples disclosed herein, a social robot and/or a telepresence robot can be used in a patient's residence to assist with patient-based monitoring and interaction.

Also, in examples disclosed herein, a patient's behavior is described as compared to a population behavioral baseline to determine the normalcy of a patient's behavior within their peer group and also as an indication that the patient does not have a particular mental illness. In other examples, the teachings of this disclosure may be adapted such that a patient's behavior is compared to a population behavioral baseline of a population exhibiting a mental illness. In such examples, a correlation of the patient's behavior with the population behavioral baseline would be indicative of the patient experiencing the mental illness. In this example, the correlation could be used to affirm a diagnosis.

Example systems, apparatus, and methods to monitor patients and validate mental illness diagnoses are disclosed herein. Further examples and combinations thereof include the following: Example 1 includes an apparatus to validate a mental illness diagnosis, the apparatus including an identifier to identify a population behavioral baseline based on a patient-specific demographic data, the patient-specific demographic data retrieved from at least one of a third-party subscriber data or an audience measurement entity data, an evaluator to compare a patient behavioral baseline to the population behavioral baseline to determine a correlation between the patient behavioral baseline and the population behavioral baseline, and a validator to identify the diagnosis as valid when the correlation is low.

Example 2 includes the apparatus of Example 1, further including a classifier to classify a patient into a demographic category based on patient data, the demographic category used to retrieve the patient-specific demographic data.

Example 3 includes the apparatus of Example 2, wherein the patient-specific demographic data includes data from an audience measurement entity panel meter.

Example 4 includes the apparatus of Example 1, wherein the validator is to determine a risk level of the patient behavior, the apparatus further including a data collector to monitor the patient, the data collector to be engaged based on a risk level of the patient behavior.

Example 5 includes the apparatus of Example 4, wherein the risk level of the patient behavior includes a risk indicative of a suicide attempt by the patient.

Example 6 includes the apparatus of Example 4, wherein the data collector is to collect at least one of a patient behavioral data, a patient physiological data, or a patient-specific environmental data.

Example 7 includes the apparatus of Example 6, further including a notifier to issue an alert when the validator identifies a safety risk to a patient, the safety risk including at least one of a deviation from the patient behavioral baseline based on the patient behavioral data, a physiological change based on the patient physiological data, or an environment-based risk based on the environmental data.

Example 8 includes the apparatus of Example 4, wherein the data collector is a wearable patient monitoring device.

Example 9 includes a method to validate a mental illness diagnosis, the method including collecting, by executing instructions with a processor, patient behavioral data during a patient assessment period, establishing, by executing instructions with the processor, a patient behavioral baseline based on the patient behavioral data, accessing, by executing instructions with the processor, patient-specific demographic data from at least one of a third-party subscriber data or an audience measurement entity data, identifying, by executing instructions with the processor, a population behavioral baseline based on the patient-specific demographic data, comparing, by executing instructions with the processor, the patient behavioral baseline to the population behavioral baseline to determine a correlation between the patient baseline and the population baseline, and identifying, by executing instructions with the processor, the diagnosis as valid when the correlation is low.

Example 10 includes the method of Example 9, wherein the patient behavioral data includes data obtained from use of a computing device.

Example 11 includes the method of Example 9, wherein the audience measurement entity data includes panel meter data derived from registered panelists.

Example 12 includes the method of Example 9, further including identifying, by executing instructions with the processor, a risk level of a patient based on the patient behavioral data, and activating, by executing instructions with the processor, a data collector to monitor the patient based on the risk level identification.

Example 13 includes the method of Example 12, wherein activating the data collector includes collecting, by executing instructions with the processor, at least one of a behavioral data, a physiological data, or an environmental data.

Example 14 includes the method of Example 13, further including identifying, by executing instructions with the processor, a safety risk to the patient based on at least one of a deviation from the patient behavioral baseline based on the behavioral data, a physiological change based on the physiological data, or an environment-based risk based on the environmental data.

Example 15 includes a non-transitory computer readable storage medium comprising computer readable instructions that, when executed, cause one or more processors to at least establish a patient behavioral baseline based on patient behavioral data collected during a patient assessment period, access patient-specific demographic data from at least one of a third-party subscriber data or an audience measurement entity data, identify a population behavioral baseline based on the patient-specific demographic data, compare the patient behavioral baseline to the population behavioral baseline to determine a correlation between the patient baseline and the population baseline, and assess the diagnosis based on the correlation.

Example 16 includes the computer readable storage medium of Example 15, wherein the instructions, when executed, cause the one or more processors to validate the diagnosis when the correlation is low.

Example 17 includes the computer readable storage medium of Example 15, wherein the instructions, when executed, further cause the one or more processors to identify a risk of patient suicide based on the patient behavioral data.

Example 18 includes the computer readable storage medium of Example 17, wherein the instructions, when executed, further cause the one or more processors to monitor the patient based on the risk identification.

Example 19 includes the computer readable storage medium of Example 18, wherein the instructions, when executed, further cause the one or more processors to collect at least one of a behavioral data, a physiological data, or an environmental data.

Example 20 includes the computer readable storage medium of Example 19, wherein the instructions, when executed, further cause the one or more processors to identify a safety risk to the patient based on at least one of a deviation from the patient behavioral baseline based on the behavioral data, a physiological change based on the physiological data, or an environment-based risk based on the environmental data.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus to validate a mental illness diagnosis, the apparatus including:
an identifier to identify a population behavioral baseline based on a patient-specific demographic data, the patient-specific demographic data retrieved from at least one of a third-party subscriber data or an audience measurement entity data;
an evaluator to compare a patient behavioral baseline to the population behavioral baseline to determine a correlation between the patient behavioral baseline and the population behavioral baseline; and
a validator to identify the diagnosis as valid when the correlation is low.

2. The apparatus of claim 1, further including a classifier to classify a patient into a demographic category based on patient data, the demographic category used to retrieve the patient-specific demographic data.

3. The apparatus of claim 2, wherein the patient-specific demographic data includes data from an audience measurement entity panel meter.

4. The apparatus of claim 1, wherein the validator is to determine a risk level of the patient behavior, the apparatus further including a data collector to monitor the patient, the data collector to be engaged based on a risk level of the patient behavior.

5. The apparatus of claim 4, wherein the risk level of the patient behavior includes a risk indicative of a suicide attempt by the patient.

6. The apparatus of claim 4, wherein the data collector is to collect at least one of a patient behavioral data, a patient physiological data, or a patient-specific environmental data.

7. The apparatus of claim 6, further including a notifier to issue an alert when the validator identifies a safety risk to a patient, the safety risk including at least one of a deviation from the patient behavioral baseline based on the patient behavioral data, a physiological change based on the patient physiological data, or an environment-based risk based on the environmental data.

8. The apparatus of claim 4, wherein the data collector is a wearable patient monitoring device.

9. A method to validate a mental illness diagnosis, the method including:
collecting, by executing instructions with a processor, patient behavioral data during a patient assessment period;
establishing, by executing instructions with the processor, a patient behavioral baseline based on the patient behavioral data;
accessing, by executing instructions with the processor, patient-specific demographic data from at least one of a third-party subscriber data or an audience measurement entity data;
identifying, by executing instructions with the processor, a population behavioral baseline based on the patient-specific demographic data;
comparing, by executing instructions with the processor, the patient behavioral baseline to the population behavioral baseline to determine a correlation between the patient baseline and the population baseline; and
identifying, by executing instructions with the processor, the diagnosis as valid when the correlation is low.

10. The method of claim 9, wherein the patient behavioral data includes data obtained from use of a computing device.

11. The method of claim 9, wherein the audience measurement entity data includes panel meter data derived from registered panelists.

12. The method of claim 9, further including:
identifying, by executing instructions with the processor, a risk level of a patient based on the patient behavioral data; and
activating, by executing instructions with the processor, a data collector to monitor the patient based on the risk level identification.

13. The method of claim 12, wherein activating the data collector includes collecting, by executing instructions with the processor, at least one of a behavioral data, a physiological data, or an environmental data.

14. The method of claim 13, further including identifying, by executing instructions with the processor, a safety risk to the patient based on at least one of a deviation from the patient behavioral baseline based on the behavioral data, a physiological change based on the physiological data, or an environment-based risk based on the environmental data.

15. A non-transitory computer readable storage medium comprising computer readable instructions that, when executed, cause one or more processors to at least:
establish a patient behavioral baseline based on patient behavioral data collected during a patient assessment period;
access patient-specific demographic data from at least one of a third-party subscriber data or an audience measurement entity data;
identify a population behavioral baseline based on the patient-specific demographic data;
compare the patient behavioral baseline to the population behavioral baseline to determine a correlation between the patient baseline and the population baseline; and
assess the diagnosis based on the correlation.

16. The computer readable storage medium of claim 15, wherein the instructions, when executed, cause the one or more processors to validate the diagnosis when the correlation is low.

17. The computer readable storage medium of claim 15, wherein the instructions, when executed, further cause the one or more processors to identify a risk of patient suicide based on the patient behavioral data.

18. The computer readable storage medium of claim 17, wherein the instructions, when executed, further cause the one or more processors to monitor the patient based on the risk identification.

19. The computer readable storage medium of claim 18, wherein the instructions, when executed, further cause the one or more processors to collect at least one of a behavioral data, a physiological data, or an environmental data.

20. The computer readable storage medium of claim 19, wherein the instructions, when executed, further cause the one or more processors to identify a safety risk to the patient based on at least one of a deviation from the patient behavioral baseline based on the behavioral data, a physiological change based on the physiological data, or an environment-based risk based on the environmental data.
